(19) United States
(12) Patent Application Publication
(10) Pub. No.: US 2025/0143610 A1
(43) Pub. Date: May 8, 2025

(54) METHOD FOR SPATIALLY CONSTRAINING ENZYME ACTIVITY

(71) Applicant: LAXMI THERAPEUTIC DEVICES, INC., Goleta, CA (US)

(72) Inventors: Ian Casson, Santa Barbara, CA (US); William Peter Van Antwerp, Santa Clarita, CA (US)

(21) Appl. No.: 18/940,377

(22) Filed: Nov. 7, 2024

Related U.S. Application Data
(60) Provisional application No. 63/596,708, filed on Nov. 7, 2023.

(51) Int. Cl. A61B 5/1486; A61B 5/145; C12N 11/14

(52) U.S. Cl. CPC A61B 5/1486; A61B 5/1451; A61B 5/14532; C12N 11/14; A61B 2562/125

(57) ABSTRACT

A method of manufacturing a sensor assembly for an analyte sensor includes, forming a substrate, forming at least one electrode on the substrate, forming an enzymatic layer on the substrate and the at least one electrode, and disabling functionality of at least part of the enzymatic layer while a portion of the enzymatic layer over at least part of the at least one electrode remains active.

### CROSS-REFERENCE TO RELATED APPLICATION(S)

The present application claims priority to and the benefit of U.S. Provisional Patent Application No. 63/596,708, filed Nov. 7, 2023, the contents of which are hereby incorporated by reference in their entirety.

### BACKGROUND

### Field

The present disclosure relates to sensors intended to be applied to a body of a subject, where the sensors may be formed or otherwise used with apparatuses that can measure monitor one or more analytes in the subject, such as the subject's glucose levels, as well as devices that incorporate such sensors. In some cases, sensor performance may be improved if the active sensor area is subject to spatial constraints and/or limitations, or are otherwise modified. The present disclosure further relates to methods of using such devices.

### Description of Related Art

Monitoring different analytes in the human body can be used for various diagnostic reasons. In particular, monitoring glucose levels is important for individuals suffering from type 1 or type 2 diabetes. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications.

Analyte monitors, and in particular, glucose monitors for the monitoring of glucose levels for the management of diabetes, are constantly being developed and improved. Although there are several platforms for monitoring analytes such as glucose available on the market, there is still a need to improve their precision, wearability, and accessibility to end-users. In addition, there is a desire to provide robust, less painful, less error-prone, and/or generally more effective continuous glucose monitors which may be attached to the patient's body for a more prolonged period of time, as well as glucose monitor features that can be used together with such improved applicators and applicator designs.

For some continuous glucose monitors, the sensors that are utilized to monitor glucose levels may be enzymatic biosensors. Enzymatic biosensors use enzymes to create a chemical reaction resulting in the oxidation of glucose in the patient's body, where a change in current or voltage potential due to the products of the oxidation reaction can be measured and converted into a glucose level reading for the patient. Sensors which are located in the interstitial fluid of a patient may utilize glucose oxidase (GOx) as the enzyme, but other enzymes can be used as well.

In some cases, particularly with continuous glucose monitors that are intended to be worn by patients for prolonged periods of time, the enzyme's function may begin to negatively affect the accuracy or other properties of the sensor, with such effects potentially being compounded over time.

### SUMMARY

Many continuous glucose monitors are intended to be worn on a patient's skin for a duration of multiple days or weeks. As noted above, most or all commercially available glucose sensors on the market today sense glucose in interstitial fluid (ISF) below the surface of the skin. Such sensing or monitoring therefore typically involves an initial step of inserting a sensor of the glucose monitor under the patient's skin. For the most part, this insertion step will involve puncturing the surface of the skin, for example, with a separate needle, for example, on an applicator, to provide access for inserting the sensor. In addition, continuous glucose monitors may include a device body that remains adhered to the patient for a prolonged period of time as well. The sensors generally work by stimulating an electrochemical reaction in a region of the sensor, for example, by using an enzyme such as GOx described above, and measuring changes in electrical parameters due to the reaction which can be used to measure the patient's blood glucose levels, such as by the use of sensor electrodes, resulting from that electrochemical reaction.

Further as noted above, particularly with continuous glucose monitors and other sensors that are intended to be used over prolonged periods of time, the reactions caused by GOx or other enzymes may have a negative effect on the sensor, and such effects may increase the longer a sensor is in use. For example, while the sensing regions of the electrodes of the sensors are essential to measuring changes in voltage or current from the reaction between the enzymes and the surrounding glucose, other parts or areas of the electrodes or the sensor in general may be damaged or otherwise affected by the function of the enzyme, potentially harming the effectiveness of the sensor. For example, GOx generates peroxide as a proxy analyte, which is necessary for sensing functionality. However, excess peroxide generation has deleterious effects on sensor performance and is a core issue for many glucose monitors, particularly continuous glucose monitors. Therefore, embodiments of the invention are directed to sensors that are made in such a way as to be capable of controlling or restricting certain byproducts of such electrochemical reactions that may impede the effectiveness of the measurements by the sensors or other performance aspects of the sensors. Embodiments of the invention are further directed to devices that incorporate such sensors, as well as methods of manufacture and/or methods of use of such sensors and/or devices.

According to an embodiment of the invention, a method of manufacturing a sensor assembly for an analyte sensor includes forming a substrate, forming at least one electrode on the substrate, forming an enzymatic layer on the substrate and the at least one electrode, and disabling functionality of at least part of the enzymatic layer while a portion of the enzymatic layer over at least part of the at least one electrode remains active.

The portion of the enzymatic layer that remains active may correspond to a sensing region of the at least one electrode.

The enzymatic layer may be formed substantially uniformly on the entire substrate.

Disabling functionality of the at least part of the enzymatic layer may include removing the at least part of the enzymatic layer. The removing of the at least part of the enzymatic layer may include utilizing a photolithography mask to isolate the at least part of the enzymatic layer from the portion of the enzymatic layer that remains active. The removing of the at least part of the enzymatic layer may instead include utilizing a laser to ablate the at least part of the enzymatic layer.

Disabling functionality of the at least part of the enzymatic layer may include inactivating the at least part of the enzymatic layer without removing the at least part of the enzymatic layer. The inactivating of the at least part of the enzymatic layer may include denaturation via photo oxidative binding of the active site to eliminate enzymatic activity of enzymes in the at least part of the enzymatic layer. The denaturation may be effected using short-wave ultraviolet light. The inactivating of the at least part of the enzymatic layer may instead include thermally inactivation and denaturation via heating enzymes in the at least part of the enzymatic layer above a characteristic denaturation temperature. The denaturation may be effected using infrared light.

The sensor assembly may be fabricated as part of a batch fabrication process together with a plurality of other sensor assemblies.

A mask may be utilized to isolate the at least part of the enzymatic layer from the portion of the enzymatic layer that remains active.

According to another embodiment of the invention, a sensor assembly for an analyte sensor includes a substrate, at least one electrode on the substrate, and an enzymatic layer on the substrate and the at least one electrode, wherein the functionality of at least part of the enzymatic layer is disabled while a portion of the enzymatic layer over at least part of the at least one electrode remains active.

The portion of the enzymatic layer that remains active may correspond to a sensing region of the at least one electrode.

The functionality of the at least part of the enzymatic layer may be disabled without removing the at least part of the enzymatic layer.

Enzymatic activity of enzymes in the at least part of the enzymatic layer may be eliminated via photo oxidative binding of the active site.

Enzymes in the at least part of the enzymatic layer may be thermally inactivated via heating above a characteristic denaturation temperature.

### BRIEF DESCRIPTION OF THE DRAWINGS

Further features and advantages of the invention will become apparent from the description of embodiments by means of the accompanying drawings. In the drawings:

FIGS. 1A and 1B schematically show a human body with a monitor including an analyte sensor according to embodiments of the invention, where the monitor is attached at different positions on the body.

FIG. 2 shows a perspective view from above an exemplary monitor including an analyte sensor according to embodiments of the invention.

FIG. 3 shows a perspective view from below the monitor of FIG. 2.

FIG. 4 schematically shows an illustrative embodiment of a sensor including a substrate with electrodes that are covered with an enzyme coating according to an embodiment of the invention.

FIGS. 5A and 5B schematically show two alternative embodiments of sensors where at least part of the enzyme coatings covering the electrodes are either selectively removed or are deactivated, according to embodiments of the invention.

FIG. 6 schematically illustrates general a batch processing approach to manufacturing sensors, and FIG. 7 schematically illustrates a further step of masking to facilitate selective removal or deactivation of parts of enzyme coatings covering the electrodes, according to embodiments of the invention.

### DETAILED DESCRIPTION

In the following detailed description, only certain embodiments of the subject matter of the present disclosure are described, by way of illustration. As those skilled in the art would recognize, the subject matter of the present disclosure may be embodied in many different forms and should not be construed as being limited to the embodiments set forth herein.

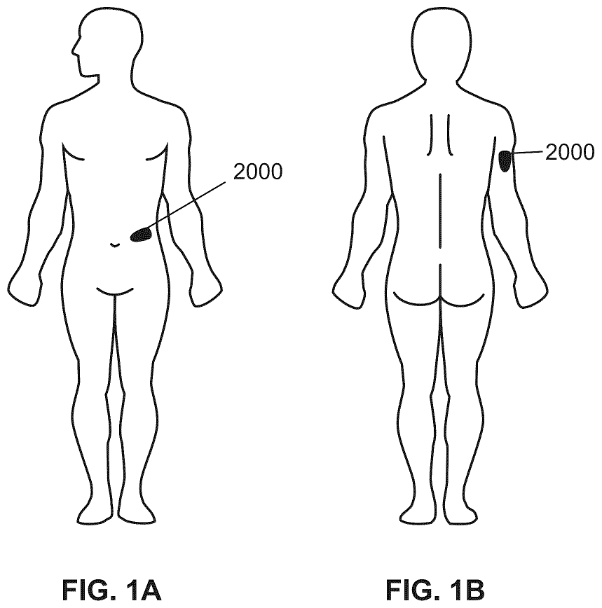

Monitors that include analyte sensors, such as glucose monitors, and particularly continuous glucose monitors, can be attached to a patient's body in different locations, in order to for example, improve glucose monitoring and/or a patient's comfort, since the continuous glucose monitors must remain adhered to the patient's skin, sometimes for a few days or more. FIG. 1A shows a first exemplary analyte monitor 2000 that is adhered to a patient's abdominal region, while FIG. 1B instead shows the exemplary analyte monitor 2000 adhered to a patient's arm. These are only meant to be example adhesion sites, and in other situations, this or a similar analyte monitor may instead be adhered or otherwise attached to other parts of the patient's body.

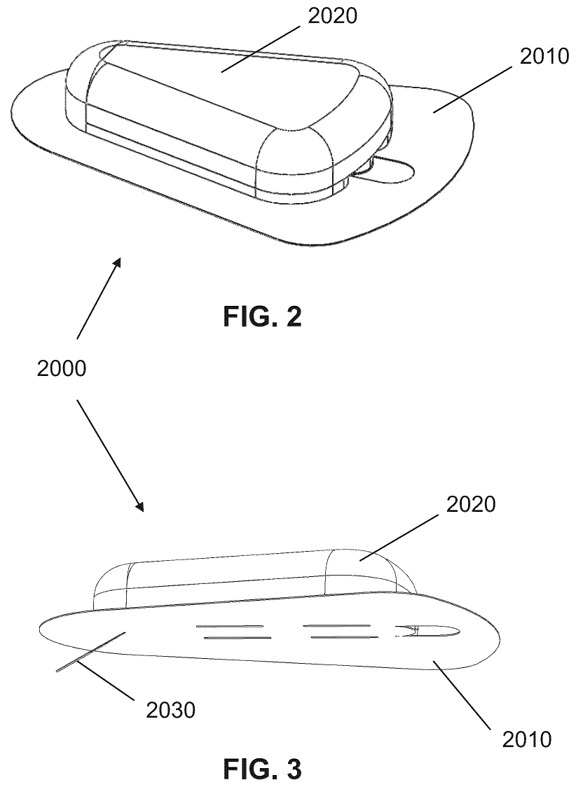

FIGS. 2 and 3 show different schematic views of an example analyte monitor 2000, which can be a continuous glucose monitor, according to an embodiment of the invention. The continuous glucose monitor 2000 may include a base or cradle 2010 that may have an adhesive layer for adhering to a patient's skin, a transmitter 2020 for transmitting data to and/or from a location away from the monitor, and a sensor member 2030 which may include an integrated analyte sensing region such as a glucose sensor. It is to be understood that the example analyte monitor shown in FIGS. 2 and 3 are for illustrative and descriptive purposes only, and that analyte monitors with different structures and functionality can also be used in conjunction with the sensor assemblies described below, without departing from the spirit or scope of the invention.

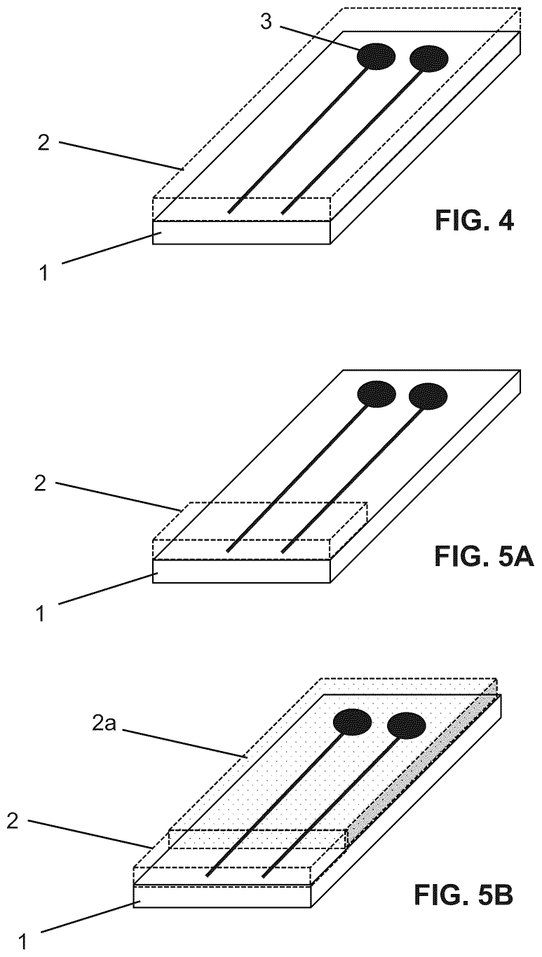

FIG. 4 provides a simplified schematic illustration of an example of a sensor intended to be inserted under a patient's skin as part of an assembly, for example, a continuous glucose monitor. For ease of description, the sensor in FIG. 4 is presented as a substantially rectangularly-shaped sensor, with relatively short electrodes provided on a substrate, as will be described in greater detail below. However, it is to be understood that the size, shape, and relative dimensions of the sensor in FIG. 4 is intended to be exemplary only, and that sensors having other shapes and proportions can also be contemplated without departing from the spirit or scope of the invention. In particular, a sensor will typically be more elongate than that shown in FIG. 4, since the sensor is intended to be inserted under a patient's skin, and so a sensor that is more elongate will allow for the sensor to be inserted under the patient's skin more readily, and for example, a narrower substrate will reduce irritation. Other shapes aside from rectangular can also be used, for example, a sensor with wings and/or a teardrop-shaped anchor, which may improve anchoring and reduce occurrences of the sensor being pulled out or dislodged from its desired position. Also, other sensors may be, for example, bent to facilitate, for example, connection with other electrical components within the monitor and/or to facilitate an angled insertion under the patient's skin. Still other sensors may be cannulated or otherwise able to facilitate drug delivery in addition to sensing.

As seen in FIG. 4, a substrate 1, which may be, for example, a flat piece of a suitable material, can be sized and shaped to facilitate implantation under a patient's skin, for example, sufficiently thin and long so as to allow for ease of insertion while minimizing or otherwise reducing irritation for the patient. In one embodiment, the sensor is thin enough to fit within the hollow core of a needle of an applicator, to facilitate transport of the sensor inside the needle when the needle pierces a patient's skin. In such cases, after the needle pierces the patient's skin, the needle may be retracted while leaving the sensor in a desired location under the patient's skin. In still other embodiments, the thickness, rigidity, and shape of the sensor can be selected to facilitate self-piercing and insertion into a patient's skin, instead of requiring a separate needle. It should therefore be understood that the relative dimensions of the sensor shown in FIG. 4 are intended to be a simplified example for ease of description, and that embodiments of the sensor according to the invention should not be limited thereto.

The embodiment of the sensor shown in FIG. 4 further includes one or more electrodes 3. Typically, there will be at least two or more electrodes 3 formed on the electrode, and in many embodiments three or more electrodes 3. Only two electrodes 3 are shown in the examples discussed herein for simplicity. The electrodes 3 can be deposited on the substrate 1 during fabrication, for example, via a known deposition process, or can be otherwise formed on or affixed to substrate 1. The electrodes 3 are configured to measure voltage and/or current changes caused by an electrochemical reaction in the blood or interstitial fluid of the patient's body in which they are inserted, for example, between glucose and GOx, and can be used to measure for example, glucose levels of the patient. To this effect, the present disclosure is primarily directed towards glucose monitoring, but the teachings herein may apply to other blood or fluid measurement applications as well.

One or more coatings 2 can then be applied to the substrate 1, including over the electrodes 3. The material or materials used for the coating may be selected to provide a protective layer over at least select regions of the electrode, and/or to stimulate chemical reactions in the region of the sensor. For example, if an enzyme such as GOx or a mixture including GOx is used for a coating, the GOx may interact with nearby glucose, which may affect the potential applied across the electrodes 3. Thus, changes in electrical parameters measurable by the sensor may take place in the region where the coating exists, whereby the measurements can then be used to approximate a glucose level of the patient. According to embodiments of the invention, the coating 2 may be applied uniformly or substantially uniformly over the entire substrate 1 during manufacturing, such that the entire electrodes 3 and surrounding area may be initially covered by the coating 2. In some other embodiments, only parts of the substrate 1, including for example, an area corresponding to the sensing region of the electrode, may instead be covered by the coating 2, while other portions of the substrate 1 may not be covered by the coating 2. However, in practice, forming a partial coating layer 2 may be more difficult to control and achieve.

As noted above, for glucose measurements, the coating may be an enzymatic material such as GOx or other similar enzyme. However, in particular with continuous glucose monitors or other monitors where use of a single sensor is desired to be longer in duration, issues that were less prevalent for single use measuring devices may become more prominent. As the desire to produce more persistent and longer lasting biosensors rises to provide more clinically accurate readings for longer device lifetimes and give patients longer-term peace of mind, such issues will need to be addressed. Most significantly, excess enzyme activity and the resultant byproducts the enzyme produces may pose many threats to the functional lifetime of an implanted biosensor. Particularly, the existence of unmitigated peroxide, which is a byproduct of the chemical reaction between glucose and GOx, outside or in excess of the detection regime, poses a threat to sensor stability in at least three significant ways. First, chemical oxidation may occur due to the produced peroxide or other byproducts, which may lead to accelerated deterioration or other destruction of the sensor materials and/or coatings. Second, peroxide is known to be an immunogenic molecule and has the potential to initiate an immune response that ultimately results in reduced sensor lifetimes when released into the tissue surrounding the sensor. Third, with prolonged use, the sensor may be more sensitive to micromovements as the sensor is moved or repositioned slightly in the tissue due, for example, to movements by the user. As the enzyme creates chemical reactions around the sensor, if the sensor moves unexpectedly, the working portions of the electrodes may shift into an area where an unexpected or undesired chemical reaction has already occurred, adding to the change in potential and temporarily inflating readings with large sudden increases in signal.

In some cases, a sacrificial protein layer may be further added to decompose peroxide that is not consumed by the electrochemical reaction. However, there is a theoretical time constraint imposed by such a coating. Over time, peroxide nevertheless deteriorates this sacrificial protein layer's ability to function as intended, which would still eventually allow resulting peroxide to escape and ultimately causing the sensor to fail in the scenarios described above.

Thus it may be advantageous to find a more effective way to restrict or otherwise regulate where and how much enzymatic coating is formed or left on the sensor, for example to reduce enzymatic coating to only targeted areas of the sensor, thereby allowing for effective electrical parameter measurements in those targeted or desired areas, while reducing chemical reactions around areas of the sensor which are not needed for an accurate measurement, and consequently reducing excessive byproducts that may be harmful to either the sensor or the patient (e.g., unnecessarily triggering an immune response in the patient or otherwise agitating the patient's body).

With spatially targeted enzyme functionalization in the electrochemical application for glucose detection, peroxide generation may only be limited or constrained to areas where the potential applied to the sensor can amperometrically detect it, while reducing peroxide generation in other non-targeted areas and thereby increasing the longevity and life span of the sensor. Forming targeted areas of active enzymatic coating as discussed above can be achieved in various ways according to embodiments of the invention. As shown in FIG. 5A, one approach can be to remove portions of the coating 2 from selected portions covering substrate 1, thereby restricting or otherwise limiting electrochemical reactions to the vicinity of the sensing regions of electrodes 3, while there may be less or no enzymatic material in other areas in order to reduce the amount of potentially harmful byproduct in undesirable areas. This may be done in a variety of ways, including targeted laser ablation or via a photolithography mask, among other methods. As shown schematically in FIG. 5A, the larger nodes near a top end of the electrodes 3, which may symbolize contacts configured to connect to a larger device such as a continuous glucose monitor, as well as a majority of the lengths of the electrodes, may have portions of the coating 2 covering them removed, while the distal or bottom regions of the electrodes 3, which may for example, symbolize the sensing regions of the electrodes 3, remain covered by the coating 2 which may contain the enzymatic material, for example.

Another approach, shown in FIG. 5B, can be to keep the entire coating 2 intact, but instead to deactivate portions of the coating 2, shown as region 2a in FIG. 5B, where enzymatic activity is not desired. Such an approach may have the same or very similar effect of limiting the reaction areas to only desired regions as the removal process described in FIG. 5A. Such can be achieved, for example, via a photolithography mask as well, in combination with, for example, short-wave ultraviolet light, as described in greater detail below. Another method of rendering portions of the enzymatic coating 2 inactive may be via thermal inactivation using infrared light, also discussed below.

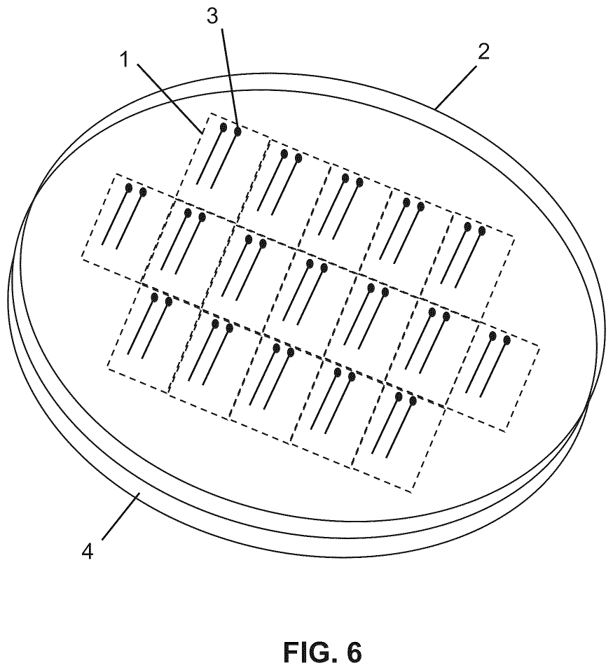

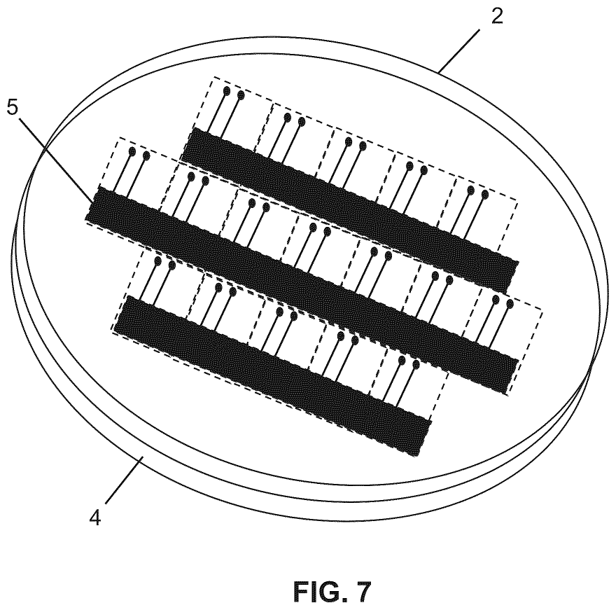

If the substrates are batch processed, as illustrated in FIGS. 6 and 7, the coating and selective targeting of active sensor portions can be accomplished effectively. For example, as shown in FIG. 6, substrates 1 with sensors 3 may be processed in batches as dies on a base, such as a polyimide wafer 4 using photolithographic techniques. Enzymatic coating layers 2 (as well as other possible layers not described herein) may be applied with spin coating or other deposition techniques.

Then, as shown in FIG. 7, targeted sensor areas may be selected and isolated with an additional mask 5. The mask can either be used to apply a protective region or an exposure region, depending on the fabrication process being used. As shown, the mask 5 covers the areas of the electrodes 3 where the enzymatic coating 2 is desired, e.g., in the functional areas of the exposed or active sensing regions near a distal end of the electrodes. The mask or similar mechanism can be used in conjunction with, for example, short-wave ultraviolet light. The mechanism of denaturation via photo oxidative binding of the active site (e.g., flavin adenine dinucleotide active site for GOx) via short-wave ultraviolet light exposure can be used to eliminate enzymatic activity in similar oxidase enzymes in regions where enzymatic reactions are not desired. Alternatively, thermal inactivation using infrared light in conjunction with a mask or similar may induce irreversible changes in conformation in enzymes that are heated above a characteristic denaturation temperature. In still other embodiments (not shown), laser ablation to remove undesired areas of coating 2 may also be employed. Infrared laser ablation can be used for cutting and shaping operations, to for example remove enzymatic coating layers in areas where reactions are undesirable, to facilitate targeted enzyme activity as well. Other embodiments may further use other techniques and methods to arrive at the same end product without departing from the spirit or scope of the invention.

In general, embodiments of the invention will include first preparing a uniform coating layer 2, and thereafter preparing a selectively functionalized enzymatic layer using the methods described above, for example, to remove or deactivate regions where enzymatic activity is not desired. With the above methods or similar methods, the coating and targeted active region selection steps can be efficiently accomplished at the batch processing part of the sensor manufacture. A sensor with targeted enzyme activity has potential for increased performance and longevity. These devices and methods provide an opportunity to fabricate sensors with longer usable life, more robust signal stability, and a more facile method of production than other processes currently available.

In addition to the embodiments that have already been described above, it is also possible to combine embodiments, e.g., different features from the various described embodiments, to provide even more different variations of targeted coating regions and sensors without departing from the spirit or scope of the invention. In addition, the inventions should not be limited to the structures and/or shapes described in the embodiments above.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present disclosure, and should not be interpreted in an idealized or overly formal sense, unless expressly so defined herein.

While the subject matter of the present disclosure has been described in connection with certain embodiments, it is to be understood that the subject matter of the present disclosure is not limited to the disclosed embodiments, but, on the contrary, the present disclosure is intended to cover various modifications and equivalent arrangements included within the spirit and scope of the appended claims, and equivalents thereof.

What is claimed is:

1. A method of manufacturing a sensor assembly for an analyte sensor, comprising:
forming a substrate;
forming at least one electrode on the substrate;
forming an enzymatic layer on the substrate and the at least one electrode; and
disabling functionality of at least part of the enzymatic layer while a portion of the enzymatic layer over at least part of the at least one electrode remains active.

2. The method of claim 1, wherein the portion of the enzymatic layer that remains active corresponds to a sensing region of the at least one electrode.

3. The method of claim 1, wherein the enzymatic layer is formed substantially uniformly on the entire substrate.

4. The method of claim 1, wherein disabling functionality of the at least part of the enzymatic layer comprises removing the at least part of the enzymatic layer.

5. The method of claim 4, wherein the removing of the at least part of the enzymatic layer comprises utilizing a photolithography mask to isolate the at least part of the enzymatic layer from the portion of the enzymatic layer that remains active.

6. The method of claim 4, wherein the removing of the at least part of the enzymatic layer comprises utilizing a laser to ablate the at least part of the enzymatic layer.

7. The method of claim 1, wherein disabling functionality of the at least part of the enzymatic layer comprises inactivating the at least part of the enzymatic layer without removing the at least part of the enzymatic layer.

8. The method of claim 7, wherein the inactivating of the at least part of the enzymatic layer comprises denaturation via photo oxidative binding of the active site to eliminate enzymatic activity of enzymes in the at least part of the enzymatic layer.

9. The method of claim 8, wherein the denaturation is effected using short-wave ultraviolet light.

10. The method of claim 7, wherein the inactivating of the at least part of the enzymatic layer comprises thermally inactivation and denaturation via heating enzymes in the at least part of the enzymatic layer above a characteristic denaturation temperature.

11. The method of claim 10, wherein the denaturation is effected using infrared light.

12. The method of claim 1, wherein the sensor assembly is fabricated as part of a batch fabrication process together with a plurality of other sensor assemblies.

13. The method of claim 1, wherein a mask is utilized to isolate the at least part of the enzymatic layer from the portion of the enzymatic layer that remains active.

14. A sensor assembly for an analyte sensor comprising:
a substrate;
at least one electrode on the substrate; and
an enzymatic layer on the substrate and the at least one electrode;
wherein the functionality of at least part of the enzymatic layer is disabled while a portion of the enzymatic layer over at least part of the at least one electrode remains active.

15. The sensor assembly of claim 14, wherein the portion of the enzymatic layer that remains active corresponds to a sensing region of the at least one electrode.

16. The sensor assembly of claim 14, wherein the functionality of the at least part of the enzymatic layer is disabled without removing the at least part of the enzymatic layer.

17. The sensor assembly of claim 14, wherein enzymatic activity of enzymes in the at least part of the enzymatic layer is eliminated via photo oxidative binding of the active site.

18. The sensor assembly of claim 14, wherein enzymes in the at least part of the enzymatic layer are thermally inactivated via heating above a characteristic denaturation temperature.
